(19) United States
(12) Patent Application Publication
(10) Pub. No.: US 2011/0117207 A1
(43) Pub. Date: May 19, 2011

(54) USE OF EGGSHELL MEMBRANE FORMULATIONS TO ALLEVIATE JOINT PAIN

(75) Inventors: John A. MINATELLI, Mt. Dora, FL (US); W. Stephen Hill, Ocala, FL (US); Michael Cielensky, Weston, FL (US)

(73) Assignee: U.S. Nutraceuticals, LLC d/b/a Valensa International State of Incorporation:, Eustis, FL (US)

(21) Appl. No.: 12/946,924

(22) Filed: Nov. 16, 2010

Related U.S. Application Data
(60) Provisional application No. 61/261,921, filed on Nov. 17, 2009.

(51) Int. Cl. A61K 35/54; A61P 29/00; A61P 19/02

(52) U.S. Cl. 424/581

(57) ABSTRACT

A method to treat and alleviate symptoms of joint pain in an individual is accomplished by administering a therapeutic amount of fowl eggshell membrane or processed eggshell membrane preparations in synergistic combination with other active constituents in an oral dosage form.

### RELATED APPLICATION

This application is based upon prior filed U.S. provisional application Ser. No. 61/261,921 filed Nov. 17, 2009.

### FIELD OF THE INVENTION

This invention relates to eggshell membrane applications, and more particularly, this invention relates to treating and alleviating symptoms of joint pain using eggshell membranes or processed eggshell membrane preparations.

### BACKGROUND OF THE INVENTION

It is known to use eggshell membrane compositions to alleviate everyday joint aches and pains, reduce joint discomfort, and improve joint mobility, flexibility and function. These eggshell membrane preparations are used to promote a normal inflammatory response and improve motion and provide some antioxidant activity.

U.S. Patent Publication Nos. 2008/0234195 and 2009/0104173 and U.S. Pat. No. 6,946,551 disclose various compositions derived from eggshell membrane that include hyaluronic acid in combination with naturally occurring constituents derived from eggshell membrane that can be used in one aspect for treating joint pain and joint problems. There are also compounds such as BiovaFlex™ as a natural joint health ingredient, which includes proteins and peptides derived from hydrolyzed, water-soluble egg membrane used for joint treatment.

### SUMMARY OF THE INVENTION

A method to treat and alleviate symptoms of joint pain in an individual is accomplished by administering a therapeutic amount of fowl eggshell membrane or processed eggshell membrane preparations in synergistic combination with other active constituents in an oral dosage form. A composition is also disclosed.

### DETAILED DESCRIPTION OF THE PREFERRED EMBODIMENTS

The present invention will now be described more fully hereinafter. This invention may, however, be embodied in many different forms and should not be construed as limited to the embodiments set forth herein. Rather, these embodiments are provided so that this disclosure will be thorough and complete, and will fully convey the scope of the invention to those skilled in the art.

A method to treat and alleviate symptoms of joint pain in an individual is disclosed and includes administering a therapeutic amount of fowl eggshell membrane or processed eggshell membrane preparations in synergistic combination with other active constituents in an oral dosage form. The eggshell membrane preparation in one example has been processed to increase water solubility or enriched to increased concentration of active substances. In another example, the formulation delivers 0.5-1000 mg of eggshell membrane or processed eggshell membrane preparation per daily dose. The preferred formulation delivers 40-500 mg eggshell membrane or processed eggshell membrane preparation per daily dose.

In yet another aspect, the formulation is supplemented with various molecular weight polymers of hyaluronic acid or sodium hyaluronate (hyaluronan) derived from microbial fermentation, eggshell or animal tissue whether in pure or crude form. For example, the formulation delivers 50-1000 mg hyaluronan per daily dose. In another aspect, hyaluronic acid is preferably derived from a biofermentation process and has a molecular weight between 0.5 and 100 kilodaltons (kDa). The hyaluronic acid has a molecular weight greater than 100 kDa in another aspect.

Astaxanthin is added in another aspect and has synergistic effects in combination with the other ingredients. The formulation is supplemented to deliver 0.1-12 mg astaxanthin per daily dose in one example. The astaxanthin is derived from Haematococcus pluvialis algae, Pfaffia, krill, or by synthetic routes, in the free diol, monoester or diester form in another example.

A natural or synthetic cyclooxygenase-1 or -2 inhibitor such as aspirin, acetaminophen, steroids, prednisone, NSAIDs, turmeric, Curcumin, boswellia and the like is added in another example. The composition also includes a gamma-linoleic acid rich oil such as from Borage (Borago officinalis L.), Safflower (Carthamus tinctorius L.) and the like, and is combined with an n-3 (omega-3) fatty acid rich oil such as from: fish oil, algae oil, flax seed oil, soybean oil, perilla seed oil, chia seed oil and the like—with the n-3 fatty acid being alpha-linolenic, stearidonic, eicosapentaenoic or docosapentaenoic acid. The composition is supplemented with collagen in any of its various forms in one example.

In another example, the composition is combined with anti-inflammatory and/or joint health promoting compounds such as preparations of: green lipped mussel (Perna canaliculus), Boswellia serrata, turmeric (Curcuma longa), stinging nettle (Urtica dioica), Andrographis, Cat's claw (Uncaria tomentosa), white willow (Salix alba), bromelain, Vitamin D, Magnesium, milk protein concentrates, fatty acid esters, methylsulfonylmethane (MSM), chondroitin sulfate, glucosamine sulfate, glucosamine hydrochloride, s-adenosyl methionine, proanthocyanidins, procyanidins or flavonoids. The ingredients are formulated in a chewable dosage form in another example, which could include a tablet or “gummy.”

In yet another example, a pharmaceutically acceptable formulation of eggshell membrane or processed eggshell membrane preparation is combined or supplemented with at least one of the following: glucosamine sulfate, chondroitin sulfate, collagen, astaxanthin, hyaluronic acid, methylsulfonylmethane, a gamma-linoleic acid or omega-3 fatty acid rich oil or cyclooxygenase inhibitor for the treatment of symptoms related to joint diseases such as, but not limited to osteoarthritis and rheumatoid arthritis.

In still another example, a dietary supplement acceptable formulation of eggshell membrane or processed eggshell membrane preparation is combined or supplemented with at least one of the following: glucosamine sulfate, chondroitin sulfate, collagen, astaxanthin, hyaluronic acid, methylsulfonylmethane, a gamma-linoleic acid or omega-3 fatty acid rich oil or cyclooxygenase inhibitor for the treatment of symptoms related to joint diseases such as, but not limited to osteoarthritis and rheumatoid arthritis.

In another example, a medical food acceptable formulation of eggshell membrane or processed eggshell membrane preparation is combined or supplemented with at least one of the following: glucosamine sulfate, chondroitin sulfate, collagen, astaxanthin, methylsulfonylmethane, a gamma-linoleic acid or omega-3 fatty acid rich oil or cyclooxygenase inhibitor for the treatment of symptoms related to joint diseases such as, but not limited to osteoarthritis and rheumatoid arthritis.

As to astaxanthin, it is beneficial and synergistic in combination with the other components, and as noted in the commonly assigned U.S. Patent Publication No. 2008/0124391, the disclosure which is hereby incorporated by reference in its entirety, astaxanthin (3,3′-dihydroxy-β,β-carotene-4,4′dione) CAS [47]-53-41, is a keto carotenoid pigment naturally accumulated via the diet in marine animals such as salmon, shrimp, red seabream and lobster and in birds such as flamingoes. Astaxanthin also occurs in certain microalgae such as Haematococcus pluvialis and in yeasts such as Phaffia species. The highest concentration, up to four percent of dry matter, occurs in Haematococcus. It can be chemically synthesized and obtained in naturally occurring stereoisomer form.

Astaxanthin, although related to other carotenoids such as beta-carotene, zeaxanthin and lutein, is a more powerful antioxidant. Astaxanthin is particularly potent in quenching singlet oxygen and has over five hundred times the ability to quench singlet oxygen as alpha-tocopherol. This antioxidant activity of astaxanthin is thought to be responsible for the wide range of health-promoting properties it exhibits, including skin and eye protection from damage by UV-light, anti-inflammatory activity, modulation or promotion of the immune response, reduction in aging processes and benefits to heart, liver, joints and prostate.

Astaxanthin is a pigment imparting the pinkish-red hue to the flesh of salmon and trout, and the coloring in the carapaces of shrimp, lobsters and crayfish. The astaxanthin molecule has two asymmetric carbons located at the 3 and 3′ positions of the cyclohexenone rings on either end of the molecule. Different enantiomers of the molecule result from how the hydroxyl groups (—OH) are attached to the carbon atoms at these centers of asymmetry. The three possible enantiomers of astaxanthin are (3R,3′R), (3S,3′S) and (3R,3′S; meso).

Free astaxanthin and its mono- and diesters from Haematococcus have optically pure (3S,3′S)-chirality. The (3S,3′S) isomer of astaxanthin is found in the skin and flesh of some salmonid fish.

Natural astaxanthin extract is an oily, viscous dark red lipophilic extract. The extract contains free astaxanthin, astaxanthin fatty acid mono-esters and astaxanthin fatty acid di-esters along with triglycerides and other lipophilic compounds. Carotenoid pigments from different sources of Haematococcus pluvialis include in some examples astaxanthin (total) 81-99% (which comprises free astaxanthin 1-5%; astaxanthin monoesters 46-79%; astaxanthin diesters 10-39%); β-carotene 0-5%; lutein 1-11%; canthaxanthin 0-5.5%; and other carotenoids 1-9%.

Naturally derived astaxanthin is typically the 35,3′S stereoisomer found in Haematococcus algae or 3R,3R′, primarily found in Phaffia yeast. Synthetic astaxanthin has a more complex stereoisomeric profile due to the non-stereo selectivity from the reaction conditions used in its manufacture. Haematococcus pluvialis contains mono and diesterified astaxanthin as the predominant forms of astaxanthin, while Phaffia and synthetically produced astaxanthin lack these esterifications.

Natural astaxanthin extracts contain astaxanthin in E and Z isomeric configurations.

Natural astaxanthin extract derived from Haematococcus pluvialis as an example includes astaxanthin stereoisomers as follows: (3S,3′S) 100%; (3S,3′R) and (3R,3′S) 0%; (3R,3′R) 0%, with the geometric isomer proportions, expressed as a percentage of the total astaxanthin, of about: E-astaxanthin 59%; 9Z-astaxanthin 15%; 13Z-astxanthin 26%, and non-astaxanthin carotenoid levels of about: 0.3% 13-carotene, 0.07% lutein, 0.3% canthaxanthin and 1.3% total other carotenoids.

It includes low molecular weight hyaluronic acid (HA) that is a major hydrodynamic component of synovial fluid and is a natural absorbent and lubricant for bones. It is excellent for bioavailability and maximizes interaction with target synovial cells and has anti-inflammatory mechanisms and down regulates pro-inflammatory mediators and neuro-peptide production and improves the symptoms of osteoarthritis. The HA's with molecular weights within the range of 0.5 to about 1 times 10⁶ DA were generally more effective in reducing indices of synovial inflammation and storing rheological properties of SF (visco-induction) than HA's with molecular weight greater than 2.3 times 10⁶ DA.

In induced uveitis, astaxanthin showed dose dependent ocular anti-inflammatory activity by suppression of NO, PGE-2 and TNF-Alpha by directly blocking NO synthase activity. It reduces C-Reactive Protein (CRP) blood levels: In human subjects with high risk levels of CRP three months of astaxanthin treatment resulted in 43% of patients serum CRP levels to drop below the risk level. Astaxanthin is so powerful it completely negates the pro-oxidant activity of Vioxx, which is known to cause cellular membrane lipid peroxidation leading to heart attack and stroke. Astaxanthin is absorbed by in vitro lens epithelial cells where it dramatically suppresses UVB induced lipid peroxidative mediated cell damage at umol/L concentrations suggesting use for the prevention of cataracts. In human trials astaxanthin at 4 mgs/day completely prevented joint pain following strenuous knee exercise when compared to untreated subjects.

Many modifications and other embodiments of the invention will come to the mind of one skilled in the art having the benefit of the teachings presented in the foregoing descriptions and the associated drawings. Therefore, it is to be understood that the invention is not to be limited to the specific embodiments disclosed, and that the modifications and embodiments are intended to be included within the scope of the dependent claims.

That which is claimed is:

1. A method to treat and alleviate symptoms of joint pain in an individual by administering a therapeutic amount of fowl eggshell membrane or processed eggshell membrane preparations in synergistic combination with other active constituents in an oral dosage form.

2. The method of claim 1, wherein said eggshell membrane preparation has been processed to increase water solubility or enriched to increased concentration of active substances.

3. The method of claim 1, wherein the formulation delivers 0.5-1000 mg of eggshell membrane or processed eggshell membrane preparation per daily dose.

4. The method of claim 1, wherein the preferred formulation delivers 40-500 mg eggshell membrane or processed eggshell membrane preparation per daily dose.

5. The method of claim 1, wherein the formulation is supplemented with various molecular weight polymers of hyaluronic acid or sodium hyaluronate (hyaluronan) derived from microbial fermentation, eggshell or animal tissue whether in pure or crude form.

6. The method of claim 5, wherein the formulation delivers 50-1000 mg hyaluronan per daily dose.

7. The method of claim 5, wherein hyaluronic acid is preferably derived from a biofermentation process and has a molecular weight between 0.5 and 100 kilodaltons (kDa).

8. The method of claim 5, wherein hyaluronic acid has a molecular weight greater than 100 kDa.

9. The method of claim 1, wherein the formulation is supplemented to deliver 0.1-12 mg astaxanthin per daily dose.

10. The method of claim 9, wherein the astaxanthin is derived from Haematococcus pluvialis algae, Pfaffia, krill, or by synthetic routes, in the free diol, monoester or diester form.

11. The method of claim 1, further comprising adding a natural or synthetic cyclooxygenase-1 or -2 inhibitor such as aspirin, acetaminophen, steroids, prednisone, NSAIDs and the like.

12. The method of claim 1, further comprising adding an n-3 (omega-3) fatty acid rich oil such as from: fish oil, algae oil, flax seed oil, soybean oil, perilla seed oil, chia seed oil and the like—with the n-3 fatty acid being alpha-linolenic, stearidonic, eicosapentaenoic or docosapentaenoic acid.

13. The method of claim 1, further comprising adding a collagen in any of its various forms.

14. The method of claim 1, further comprising adding anti-inflammatory and/or joint health promoting compounds such as preparations of: green lipped mussel (Perna canaliculus), Boswellia serrata, turmeric (Curcuma longa), stinging nettle (Urtica dioica), Andrographis, Cat's claw (Uncaria tomentosa), white willow (Salix alba), bromelain, Vitamin D, Magnesium, milk protein concentrates, fatty acid esters, methylsulfonylmethane (MSM), chondroitin sulfate, glucosamine sulfate, glucosamine hydrochloride, s-adenosyl methionine.

15. The method of claim 1, wherein the ingredients are formulated in a chewable dosage form such as a tablet, gelatin or pectin based “gummy” or the like.

16. A pharmaceutically acceptable formulation of eggshell membrane or processed eggshell membrane preparation combined or supplemented with at least one of the following: glucosamine sulfate, chondroitin sulfate, collagen, astaxanthin, hyaluronic acid, methylsulfonylmethane, a gamma-linoleic acid or omega-3 fatty acid rich oil or cyclooxygenase inhibitor for the treatment of symptoms related to joint diseases such as, but not limited to osteoarthritis and rheumatoid arthritis.

17. A dietary supplement acceptable formulation of eggshell membrane or processed eggshell membrane preparation combined or supplemented with at least one of the following: glucosamine sulfate, chondroitin sulfate, collagen, astaxanthin, hyaluronic acid, methylsulfonylmethane, a gamma-linoleic acid or omega-3 fatty acid rich oil or cyclooxygenase inhibitor for the treatment of symptoms related to joint diseases such as, but not limited to osteoarthritis and rheumatoid arthritis.

18. A medical food acceptable formulation of eggshell membrane or processed eggshell membrane preparation combined or supplemented with at least one of the following: glucosamine sulfate, chondroitin sulfate, collagen, astaxanthin, methylsulfonylmethane, a gamma-linoleic acid or omega-3 fatty acid rich oil or cyclooxygenase inhibitor for the treatment of symptoms related to joint diseases such as, but not limited to osteoarthritis and rheumatoid arthritis.

19. A dietary supplement formulation consisting of 100-500 mg of hydrolyzed eggshell membrane preparation, 10-40 mg sodium hyaluronate (<100 kDa), 0.5-12 mg Astaxanthin, Boswellia serrata preparation providing at least 30-100 mg Boswellic acids, and optionally containing at least 200 IU Vitamin D and/or at least 20% US RDI Magnesium and/or 100-2000 mg curcuminoids per serving.

20. A dietary supplement formulation consisting of: 100-500 mg of hydrolyzed eggshell membrane preparation, 0.5-12 mg Astaxanthin, at least 200 IU Vitamin D and/or 50-1000 mg curcuminoids per serving in a chewable delivery system such as a “gummy.”
